(19) United States
(12) Patent Application Publication
(10) Pub. No.: US 2025/0270382 A1
(43) Pub. Date: Aug. 28, 2025

(54) METHODS OF CHEMICAL DECONSTRUCTION AND UPCYCLING OF POLYETHYLENE TEREPHTHALATE

(71) Applicant: UT-Battelle, LLC, Oak Ridge, TN (US)

(72) Inventors: Jeff Foster, Knoxville, TN (US); Tomonori Saito, Knoxville, TN (US)

(21) Appl. No.: 19/059,410

(22) Filed: Feb. 21, 2025

Related U.S. Application Data
(60) Provisional application No. 63/556,418, filed on Feb. 22, 2024.

(51) Int. Cl. C08J 11/28; C07C 67/02; C08G 63/547; C08G 63/91

(52) U.S. Cl. CPC C08J 11/28; C07C 67/02; C08G 63/547; C08G 63/91; C08J 2367/02

(57) ABSTRACT

A method of deconstructing and upcycling polyethylene terephthalate (PET) waste into at least one useful breakdown product, the method comprising contacting the PET waste (1) with a catalyst comprising an organic nitrogen-containing base and a carboxylic acid or ester thereof, in the presence of an R-functionalized alcohol molecule (2) under transesterification conditions to produce a terephthalate difunctionalized monomer (3), according to the following reaction scheme:

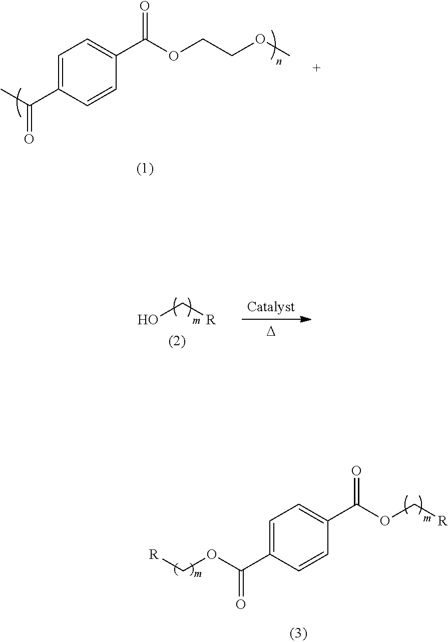

wherein: R is a functional group selected from alkenyl, alkynyl, N₃, CN, OR^{b}, COOH, CHO, C(O)R^{b}, C(O)NHR^{b}, NHC(O)NHR^{b}, OCONHR^{b}, SR^{b}, SSR^{b}, and B(OH)₂ groups, wherein R^{b} is an alkyl group containing 1-3 carbon atoms; n is at least 10; m is an integer from 1-12; and the symbol Δ denotes an elevated temperature sufficient to promote a transesterification reaction between (1) and (2) in the presence of the catalyst.

### CROSS REFERENCE TO RELATED APPLICATION

The present application claims benefit of U.S. Provisional Application No. 63/556,418, filed on Feb. 22, 2024, all of the contents of which are incorporated herein by reference.

### STATEMENT REGARDING FEDERALLY SPONSORED RESEARCH

This invention was made with government support under Prime Contract No. DE-AC05-000R22725 awarded by the U.S. Department of Energy. The government has certain rights in the invention.

### FIELD OF THE DISCLOSURE

The present disclosure generally relates to methods of breaking down and recycling plastic waste. The present disclosure more particularly relates to methods of deconstructing polyethylene terephthalate (PET) waste into useful monomeric and polymeric products.

### BACKGROUND

Plastics are critical in facilitating the comfort and quality of everyday life. Most plastics are discarded after a single use, wasting the energy and carbon consumed for their production and incurring environmental costs. The production of commodity polymers in the U.S. alone requires an estimated 3.2 quadrillion BTUs of energy annually and generates 104 MMT CO2e of greenhouse gas (GHG) emissions. A 2018 study by the EPA revealed that 76% of produced plastic mass is landfilled annually, inevitably wasting the feedstock resources, energy, and carbon used for their production.

Thus, efficient processes for plastics recycling and upcycling are needed to mitigate these losses and to combat the accumulation of plastic waste in the environment, which is now estimated to be on the order of 108 tons of used, non-biodegradable material. Thus, closed-loop production and recycling processes are needed to mitigate energy and carbon loss toward a net-zero carbon economy. However, although much effort has been made in this respect, efficient and low cost processes for recycling and upcycling of plastic waste remains a significant challenge. Thus, there would be a significant benefit in a method that can efficiently deconstruct and upcycle plastic waste, particularly PET, considering the ubiquitous nature of PET in plastic waste.

### SUMMARY OF THE DISCLOSURE

The present disclosure is directed to a novel method for deconstructing and upcycling polyethylene terephthalate (PET) waste into at least one useful monomeric or polymeric product.

The method disclosed herein is advantageously cost efficient, straight-forward, and capable of being scaled up to make it industrially feasible.

More particularly, the method includes the steps of: contacting the PET waste (1) with a catalyst comprising an organic nitrogen-containing base and a carboxylic acid or ester thereof, in the presence of an R-functionalized alcohol molecule (2) under transesterification conditions to produce a terephthalate difunctionalized monomer (3), according to the following reaction scheme:

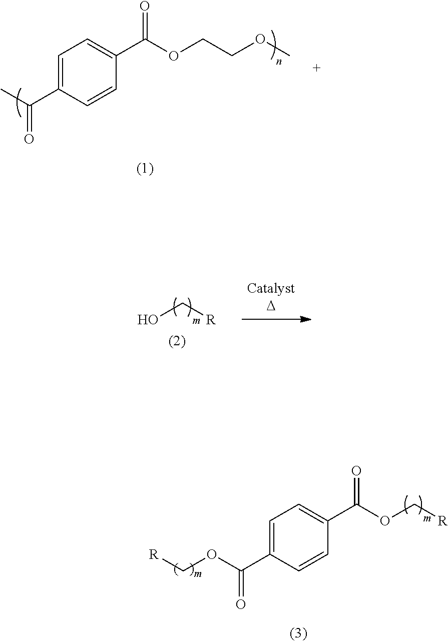

In the above scheme, R is a functional group selected from alkenyl, alkynyl, N₃, CN, OR^{b}, COOH, CHO, C(O)R^{b}, C(O)NHR^{b}, NHC(O)NHR^{b}, OCONHR^{b}, SR^{b}, SSR^{b}, and B(OH)₂ groups, wherein R^{b} is an alkyl group containing 1-3 carbon atoms; n is at least 10; m is an integer from 1-12; and the symbol Δ denotes an elevated temperature sufficient to promote a transesterification reaction between (1) and (2) in the presence of the catalyst.

In some embodiments, the functionalized alcohol molecule (2) is a hydroxy alkene molecule of the formula:

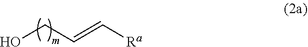

and the terephthalate difunctionalized monomer (3) is a dialkene monomer having the following formula:

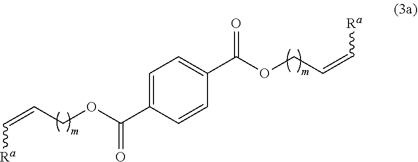

wherein: R^{a} is selected from H and alkyl groups containing 1-6 carbon atoms; m is an integer from 1-12; and the wavy line connecting to R^{a} indicates that R^{a} may be in a cis or trans position.

In some embodiments, the method further comprises polymerizing the dialkene monomer (3a) by an acyclic diene metathesis (ADMET) polymerization process to produce the following polymer:

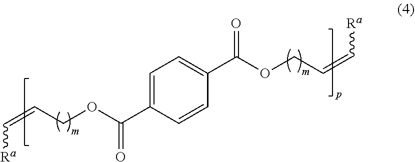

wherein: R^{a} is selected from H and alkyl groups containing 1-6 carbon atoms; m is an integer from 1-6; p is at least 10; and the wavy line connecting to R^{a} indicates that R^{a} may be in a cis or trans position.

As further shown later in this disclosure, by use of the novel methods described herein, poly(ethylene terephthalate) (PET) waste can be efficiently deconstructed into small molecule α,ω-dialkenenyl terephthalates using organocatalyzed transesterification. The resulting compounds can be polymerized, such as by acyclic diene metathesis (ADMET) polymerization, affording unsaturated semi-aromatic polyesters with thermomechanical properties dependent on the monomer structure and the catalyst used for their synthesis. High molecular weight ADMET polymers form free-standing films that are ductile and tough with mechanical properties similar to widely used commodity plastics. The obtained semi-aromatic polyester materials exhibited mechanical properties intermediate of PET and PE, both of which were strongly dependent on monomer structure and the selection of Ru initiator used for their synthesis. Significantly, the ADMET polymers can be deconstructed to monomers using Retro-ADMET, such as by using 1-hexene (i.e., hexenolysis), and subsequently re-polymerized by ADMET polymerization, thus establishing closed-loop circularity for this unique class of materials.

### BRIEF DESCRIPTION OF THE FIGURES

FIGS. 1A-1C. PET deconstruction to new monomers and their closed-loop polymerization and recycling. FIG. 1A is a schematic of PET deconstruction using ω-unsaturated alcohols and subsequent closed-loop production and recycling established via ADMET polymerization and Retro-ADMET depolymerization. FIG. 1B provides examples of α,ω-dialkenenyl terephthalate compounds and their yields synthesized via PET deconstruction by ω-unsaturated alcohols. FIG. 1C compares the ¹H NMR spectra of DHT synthesized using either terephthaloyl chloride or PET as the terephthalate source.

FIGS. 2A-2D. Polymerization of α,ω-diene monomers and characterization of the resulting polymers. FIG. 2A is a schematic showing ADMET polymerization of DnT monomers. FIG. 2B is graph containing an overlay of MW distributions for poly(DHT) synthesized using G1 or G2. FIG. 2C is a graph containing stacked DSC traces for the same poly(DHT) polymers. The solid lines correspond with poly(DHT-1), while the dashed lines to poly(DHT-2). FIG. 2D is a plot of the glass transition temperature, T_{g}, and melting temperature, Tₘ, against n for polymers synthesized using either G1 or G2 and different monomers. The symbols are assigned as follows: o=poly(DnT-1) Tₘ values, ●=poly(DnT-1) T_{g} values, ▪=poly(DnT-2) T_{g} values, where n represents the length of the methylene spacer in the repeat unit. The triangle symbols represent literature Tₘ (Δ) and T_{g} (▴) values for fully saturated polymer analogs.

FIGS. 3A-3C. Evaluation of poly(DHT) mechanical properties. FIG. 3A is a graph comparing thermal stability of poly(DHT) with commodity polymers. FIG. 3B is a graph comparing thermal transitions of poly(DHT) with commodity polymers as measured by DSC. FIG. 3C is a graph comparing tensile data of high MW poly(DHT) samples with commodity materials. Solid lines in each panel correspond to LDPE, dashed lines to PET, dotted/dashed lines to poly(DHT-1), and dotted lines to poly(DHT-2).

FIGS. 4A-4C. Deconstruction of poly(DHT-2) via Retro-ADMET. FIG. 4A is a schematic of closed-loop production and recycling of poly(DHT-2) using olefin metathesis chemistry. FIG. 4B shows overlaid ¹H NMR spectra of poly(DHT-2), and re-polymerized poly(DHT-2) that was synthesized directly from the deconstruction mixture. FIG. 4C is a graph comparing SEC RI traces showing poly(DHT-2) before deconstruction (solid line), the resulting deconstruction mixture (dotted line), and poly(DHT-2) after repolymerization (dashed line).

FIGS. 5A-5D. Hydrogenation of poly(DHT) produces fully saturated polyesters. FIG. 5A is a schematic of hydrogenation of poly(DHT) using tosyl hydrazide. FIG. 5B is a graph containing overlaid ¹H NMR spectra highlighting the disappearance of backbone olefins of poly(DHT-2) following hydrogenation. FIG. 5C is a graph containing stacked DSC traces showing the thermal behavior of the various samples before and after hydrogenation. FIG. 5D is a graph showing that overlaid TGA curves for the same samples show an increase in thermal stability following hydrogenation. The legend in FIG. 5C also applies to FIG. 5D.

### DETAILED DESCRIPTION

In a first aspect, the present disclosure is directed to a method of deconstructing and upcycling polyethylene terephthalate (PET) waste into at least one useful monomeric product. In the method, PET waste (1) is contacted with a catalyst containing an organic nitrogen-containing base and a carboxylic acid or ester thereof, in the presence of an R-functionalized alcohol molecule (2) under transesterification conditions to produce a terephthalate difunctionalized monomer (3), according to the following reaction scheme:

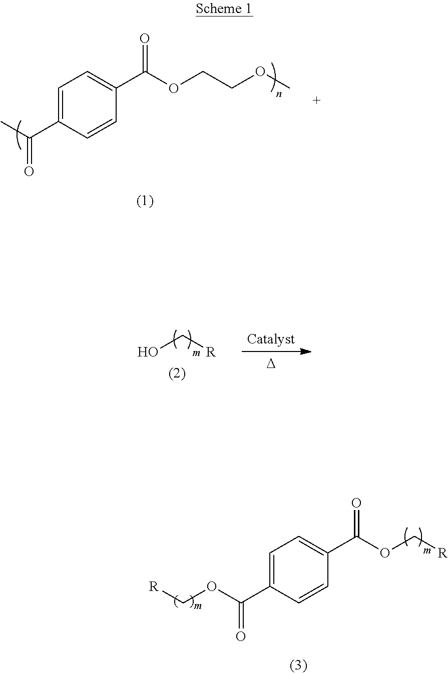

The variable R in the above Scheme 1 is or contains a functional group selected from alkenyl groups, alkynyl groups, N₃, CN, OR^{b}, COOH, CHO, C(O)R^{b}, C(O)NHR^{b}, NHC(O)NHR^{b}, OCONHR^{b}, SR^{b}, SSR^{b}, and B(OH)₂ groups, wherein R^{b} is an alkyl group containing 1-3 carbon atoms. Some examples of R^{b} groups include methyl, ethyl, n-propyl, and isopropyl groups. Alkenyl and alkynyl groups contain two or more carbon atoms, such as 2, 3, 4, 5, or 6 carbon atoms or a number of carbon atoms within a range therein (e.g., 2-6 or 2-4). In the case where R is an alkenyl or alkynyl group, the alkenyl or alkynyl group may correspond to the formula —CH═CHR′ or —C≡CR′, respectively, wherein R′ is H or a hydrocarbon group containing 1-4 carbon atoms (e.g., methyl, ethyl, n-propyl, isopropyl, n-butyl, isobutyl, sec-butyl, and t-butyl groups).

The variable n is at least 10, 20, 30, 40, 50, or 100. The variable m is an integer of 1, 2, 3, 4, 5, 6, 7, 8, 9, 10, 11, or 12 or an integer within a range bounded by any two of the foregoing values, such as 1-12, 1-10, 1-8, 1-6, 1-4, 1-3, 2-12, 2-10, 2-8, 2-6, 2-4, 3-12, 3-10, 3-8, or 3-6.

The symbol Δ in the above Scheme 1 denotes an elevated temperature sufficient to promote a transesterification reaction between (1) and (2) in the presence of the catalyst to produce the monomer (3). The elevated temperature is typically at least 150° C. and up to 250° C., provided that the temperature is below the decomposition temperature of the PET waste. In different embodiments, the temperature may be, for example, 150° C., 160° C., 170° C., 180° C., 190° C., 200° C., 210° C., 220° C., 230° C., 240° C., or 250° C., or a temperature within a range bounded by any two of the foregoing values (e.g., 150-250° C., 150-200° C., 160-250° C., 160-200° C., 170-250° C., 170-200° C., or 170-190° C.). The process may be conducted at any of the foregoing temperatures for a period of time of, for example, 1 hour, 2 hours, 3 hours, or 4 hours, or a period of time within a range therein. The method may be conducted at ambient pressure (about 1 atm) but may, in some embodiments, subject the combined components (polymer waste in contact with catalyst and functionalized alcohol) to a reduced pressure (e.g., less than 1 or 0.1 atm) or an elevated pressure, e.g., above 1 atm, or at least 2, 5, 10, 20, or 50 atm.

The catalyst used in the above Scheme 1 contains an organic nitrogen-containing base (i.e., “base” or “B species”) and a carboxylic acid or ester thereof (i.e., “acid” or “A species”). The base is typically complexed with the acid in the catalyst. The carboxylic acid or ester (Δ) species may be combined with the base (B) species in any desired B:A molar ratio, wherein the B:A molar ratio may have a B molar value and A molar value independently selected from, for example, 0.05, 0.06, 0.07, 0.08, 0.09, 0.1, 0.2, 0.25, 0.3, 0.4, 0.5, 0.6, 0.7, 0.8, 0.9, and 1. The B:A molar ratio may also be within a range of B:A molar ratios, such as a range of 0.05:1 to 1:0.05 (1:20 to 20:1). In other embodiments, the B:A molar ratio is within a range of 0.2:1 to 1:0.2 (or 1:5 to 5:1). In other embodiments, the B:A molar ratio is within a range of 0.5:1 to 1:0.5 (or 1:2 to 2:1). In other embodiments, the B:A molar ratio is about 1:1.

In embodiments, the organic nitrogen-containing base has the following structure:

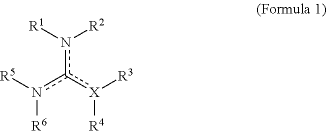

In Formula (1), the variables R¹, R², R³, R⁴, R⁵, and R⁶ are independently selected from hydrogen atom, electron pair, and alkyl groups containing one to three carbon atoms. Examples of alkyl groups containing one to three carbon atoms include methyl, ethyl, n-propyl, and isopropyl. The dotted lines represent optional double bonds. If a nitrogen atom in Formula (1) or sub-formula thereof is engaged in a carbon-nitrogen double bond, a substituent shown attached to the nitrogen atom is an electron pair to maintain charge neutrality. The variable X is C, CR^{a}, or N, wherein R^{a} is selected from hydrogen atom and alkyl groups containing one to three carbon atoms. Notably, by the rules of chemistry, the variable R¹, R², R³, R⁴, R⁵, or R⁶ can only be an electron pair when the electron pair is attached to a nitrogen atom engaged in a carbon-nitrogen double bond. Similarly, by the rules of chemistry, X is C only when C is engaged in a carbon-carbon double bond, and X is CR^{a} only when the carbon atom in CR^{a} is engaged exclusively in carbon-carbon single bonds. In some embodiments, all of R¹, R², R³, R⁴, R⁵, and R⁶ are hydrogen atoms. In other embodiments, one, two, three, four, five, or all of R¹, R², R³, R⁴, R⁵, and R⁶ are alkyl groups containing one to three carbon atoms, or more particularly, one, two, three, four, five, or all of R¹, R², R³, R⁴, R⁵, and R⁶ are selected from methyl groups, ethyl groups, n-propyl groups, or isopropyl groups or selected from a combination thereof.

In a first set of embodiments, R¹ and R² optionally interconnect to form a five, six, or seven-membered ring. In some embodiments, R¹ and R² interconnect to form a five, six, or seven-membered ring, while in other embodiments, R¹ and R² do not interconnect.

In a second set of embodiments, R² and R³ optionally interconnect to form a five, six, or seven-membered ring. In some embodiments, R² and R³ interconnect to form a five, six, or seven-membered ring, while in other embodiments, R² and R³ do not interconnect.

In a third set of embodiments, R³ and R⁴ optionally interconnect to form a five, six, or seven-membered ring. In some embodiments, R³ and R⁴ interconnect to form a five, six, or seven-membered ring, while in other embodiments, R³ and R⁴ do not interconnect.

In a fourth set of embodiments, R⁴ and R⁶ optionally interconnect to form a five, six, or seven-membered ring. In some embodiments, R⁴ and R⁶ interconnect to form a five, six, or seven-membered ring, while in other embodiments, R⁴ and R⁶ do not interconnect.

In a fourth set of embodiments, R⁵ and R⁶ optionally interconnect to form a five, six, or seven-membered ring. In some embodiments, R⁵ and R⁶ interconnect to form a five, six, or seven-membered ring, while in other embodiments, R⁵ and R⁶ do not interconnect.

In a fifth set of embodiments, R¹ and R⁵ optionally interconnect to form a five, six, or seven-membered ring. In some embodiments, R¹ and R⁵ interconnect to form a five, six, or seven-membered ring, while in other embodiments, R¹ and R⁵ do not interconnect.

Any two of the above first to fifth embodiments may be combined to result in a bicyclic compound within the scope of Formula (1). Any three of the above first to fifth embodiments may be combined to result in a tricyclic compound within the scope of Formula (1).

In some embodiments of Formula (1), the organic nitrogen-containing base has the following structure:

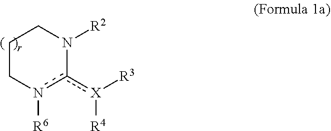

In Formula (1a), R², R³, R⁴, R⁶ and X are as defined earlier above, and the variable r is a value of 0, 1, or 2. When r is 0, a five-membered ring results. When r is 1, a six-membered ring results. When r is 2, a seven-membered ring results. In some embodiments of Formula (1a), X is C or CR^{a}. In other embodiments of Formula (1a), X is N. In some embodiments, all of R², R³, R⁴, R⁶ are hydrogen atoms. In other embodiments, one, two, three, or all of R², R³, R⁴, R⁶ are alkyl groups containing one to three carbon atoms, or more particularly, one, two, three, or all of R², R³, R⁴, R⁶ are selected from methyl groups, ethyl groups, n-propyl groups, or isopropyl groups or selected from a combination thereof. In the case where a nitrogen atom in Formula (1a) is attached to a hydrogen atom or alkyl group, the nitrogen atom is not engaged in a double bond since the presence of a double bond in such circumstance would result in a positively charged molecule, but molecules of Formula (1) and sub-formulas thereof are uncharged (neutral) for purposes of the present disclosure. If a nitrogen atom in Formula (1) or sub-formula thereof is engaged in a carbon-nitrogen double bond, the substituent (e.g., R⁴ or R⁶) is an electron pair in that case to maintain charge neutrality (no charge). In some embodiments, R⁶ is an electron pair and the nitrogen atom attached to R⁶ is engaged in a carbon-nitrogen double bond, in which case the remaining optional double bond shown in Formula (1a) is not present. In further or alternative embodiments, R⁴ is an alkyl group, such as any of the alkyl groups provided above, particularly methyl or ethyl. In further or alternative embodiments, R² and R³ optionally interconnect to form a five, six, or seven-membered ring. In further or alternative embodiments, R³ and R⁴ optionally interconnect to form a five, six, or seven-membered ring. In further or alternative embodiments, R⁴ and R⁶ optionally interconnect to form a five, six, or seven-membered ring.

In some embodiments of Formula (1a), the organic nitrogen-containing base has the following structure:

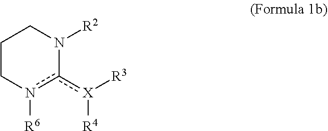

In Formula (1b), R², R³, R⁴, R⁶ and X are as defined earlier above. In some embodiments of Formula (1b), X is C or CR^{a}. In other embodiments of Formula (1b), X is N. In some embodiments, all of R², R³, R⁴, R⁶ are hydrogen atoms. In other embodiments, one, two, three, or all of R², R³, R⁴, R⁶ are alkyl groups containing one to three carbon atoms, or more particularly, one, two, three, or all of R², R³, R⁴, R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1a) is not present. In further or alternative embodiments, R⁴ is an alkyl group, such as any of the alkyl groups provided above, particularly methyl or ethyl. In further or alternative embodiments, R² and R³ optionally interconnect to form a five, six, or seven-membered ring. In further or alternative embodiments, R³ and R⁴ optionally interconnect to form a five, six, or seven-membered ring. In further or alternative embodiments, R⁴ and R⁶ optionally interconnect to form a five, six, or seven-membered ring.

In some embodiments of Formula (1), the organic nitrogen-containing base has the following structure:

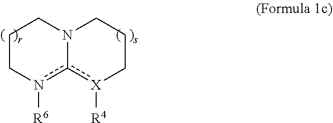

In Formula (1c), R⁴, R⁶ and X are as defined earlier above, and the variables r and s are each independently a value of 0, 1, or 2. When r or s is 0, a five-membered ring results. When r or s is 1, a six-membered ring results. When r or s is 2, a seven-membered ring results. In different embodiments, r and s are both 0; or r and s are both 1; or r and s are both 2; or r is 0 and s is 1; or r is 0 and s is 2; or r is 1 and s is 0; or r is 1 and s is 2; or r is 2 and s is 0; or r is 2 and s is 1. In some embodiments of Formula (1c), X is C or CR^{a}. In other embodiments of Formula (1c), X is N. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1c) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1c), the organic nitrogen-containing base has the following structure:

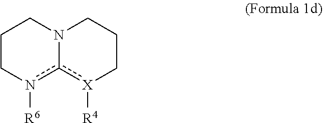

In Formula (1d), R⁴, R⁶ and X are as defined earlier above. In some embodiments of Formula (1d), X is C or CR^{a}. In other embodiments of Formula (1d), X is N. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1d) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1c), the organic nitrogen-containing base has the following structure:

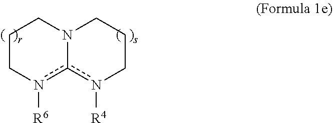

In Formula (1e), R⁴ and R⁶ are as defined earlier above, and the variables r and s are each independently a value of 0, 1, or 2. When r or s is 0, a five-membered ring results. When r or s is 1, a six-membered ring results. When r or s is 2, a seven-membered ring results. In different embodiments, r and s are both 0; or r and s are both 1; or r and s are both 2; or r is 0 and s is 1; or r is 0 and s is 2; or r is l and s is 0; or r is 1 and s is 2; or r is 2and s is 0; or r is 2and sis 1. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1e) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1e), the organic nitrogen-containing base has the following structure:

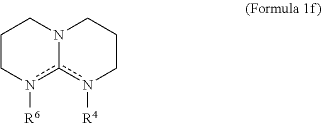

In Formula (1f), R⁴ and R⁶ are as defined earlier above. In some embodiments, R⁴ and R⁶ are hydrogen atoms. In other embodiments, one or both of R⁴ and R⁶ are alkyl groups containing one to three carbon atoms, or more particularly, one or both of R⁴ and R⁶ are selected from methyl groups, ethyl groups, n-propyl groups, or isopropyl groups or selected from a combination thereof. In some embodiments, R⁶ is an electron pair and the nitrogen atom attached to R⁶ is engaged in a carbon-nitrogen double bond, in which case the remaining optional double bond shown in Formula (1f) is not present. In further or alternative embodiments, R⁴ is an alkyl group, such as any of the alkyl groups provided above, particularly methyl or ethyl. In further or alternative embodiments, R⁴ and R⁶ optionally interconnect to form a five, six, or seven-membered ring.

In some embodiments of Formula (1), the organic nitrogen-containing base has the following structure:

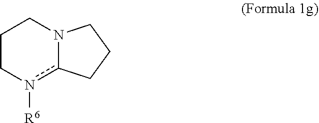

In Formula (1g), R⁶ is as defined earlier above. In some embodiments, R⁶ is a hydrogen atom. In other embodiments, R⁶ is an alkyl group containing one to three carbon atoms, or more particularly, R⁶ is selected from methyl groups, ethyl groups, n-propyl groups, or isopropyl groups or selected from a combination thereof. In the case where R⁶ is a hydrogen atom or alkyl group, the optional double bond is not present. In some embodiments, R⁶ is an electron pair and the nitrogen atom attached to R⁶ is engaged in a carbon-nitrogen double bond.

In some embodiments of Formula (1), the organic nitrogen-containing base has the following structure:

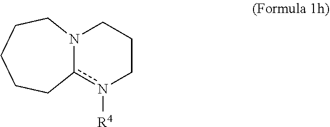

In Formula (1h), R⁴ is as defined earlier above. In some embodiments, R⁴ is a hydrogen atom. In other embodiments, R⁴ is an alkyl group containing one to three carbon atoms, or more particularly, R⁴ is selected from methyl groups, ethyl groups, n-propyl groups, or isopropyl groups or selected from a combination thereof. In the case where R⁴ is a hydrogen atom or alkyl group, the optional double bond is not present. In some embodiments, R⁴ is an electron pair and the nitrogen atom attached to R⁴ is engaged in a carbon-nitrogen double bond.

In some embodiments of Formula (1), the organic nitrogen-containing base has the following structure:

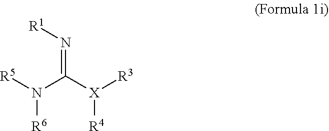

In Formula (1i), R¹, R³, R⁴, R⁵, R⁶ and X are as defined earlier above, except that R¹, R³, R⁴, R⁵, and R⁶ are not permitted to interconnect. In some embodiments of Formula (1i), X is CR^{a}. In other embodiments of Formula (1i), X is N. In some embodiments, all of R¹, R³, R⁴, R⁵, R⁶ are hydrogen atoms. In other embodiments, one, two, three, four, or all of R¹, R³, R⁴, R⁵, R⁶ are alkyl groups containing one to three carbon atoms, or more particularly, one, two, three, four, or all of R¹, R³, R⁴, R⁵, R⁶ are selected from methyl groups, ethyl groups, n-propyl groups, or isopropyl groups or selected from a combination thereof. In some embodiments, R¹, R³, and R⁵ are selected from alkyl groups containing one to three carbon atoms, such as any of the alkyl groups provided above, or R¹, R³, and R⁵ are methyl groups.

Some specific examples of organic nitrogen-containing bases include:

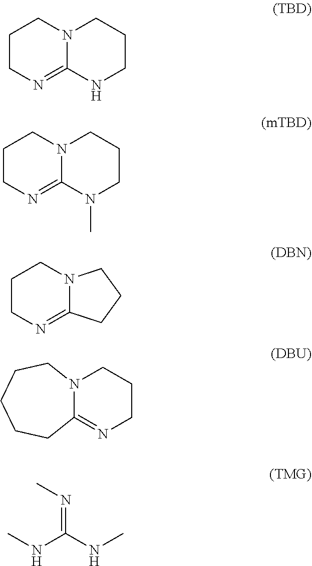

As indicated earlier above, the base, which may have any of the structures within Formulas (1), (1a), (1b), (1c), (1d), (1e), (1f), (1h), (1i), or specific structures provided above, is in combination with (typically, complexed with) a carboxylic acid or ester thereof in the catalyst. The base and acid can be included in the catalyst in any suitable molar ratio, such as any of those provided earlier above. Moreover, any base described above can be combined with any acid described in this disclosure to provide a catalyst.

The carboxylic acid or ester thereof may have the following structure:

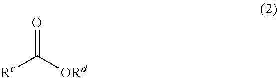

In Formula (2), R′ is a hydrogen atom (H) or a hydrocarbon group containing 1-12 carbon atoms. In different embodiments, the hydrocarbon group R^{C} contains, for example, 1, 2, 3, 4, 5, 6, 7, 8, 9, 10, 11, or 12 carbon atoms, or a number of carbon atoms within a particular range bounded by any two of the foregoing carbon numbers (e.g., 1-12, 1-8, 1-6, 1-5, 1-4, 1-3, 2-12, 2-8, 2-6, 2-5, 2-4, or 2-3 carbon atoms). As used herein, the term “hydrocarbon group” is defined as a chemical group containing at least carbon and hydrogen atoms. In some embodiments, R^{c} is composed solely of carbon and hydrogen. In other embodiments, R′ is composed of carbon and possibly hydrogen atoms except that the hydrocarbon group may (i.e., optionally) be substituted with one or more halogen (e.g., fluorine, chlorine, bromine, or iodine) atoms to result in partial or complete halogenation of the hydrocarbon group. The group R^{c} is typically a linear, branched, or cyclic alkyl group or an aromatic (e.g., phenyl) group. Some examples of linear, branched, and cyclic alkyl groups include methyl, ethyl, n-propyl, isopropyl, cyclopropyl, n-butyl, isobutyl, sec-butyl, t-butyl, cyclobutyl, n-pentyl, cyclopentyl, isopentyl, n-hexyl, cyclohexyl, n-heptyl, n-octyl, isooctyl, n-nonyl, n-decyl, n-undecyl, and n-dodecyl groups, as well as fluorinated and chlorinated versions of such groups.

In Formula (2), R^{d} is selected from H and alkyl groups containing 1-3 carbon atoms. In one set of embodiments, R^{d} is a hydrogen atom, and R^{c} may be any of the linear, branched, or cyclic alkyl groups (or a phenyl group) as provided above. In another set of embodiments, R^{d} is an alkyl group, and R^{c} may be any of the linear, branched, or cyclic alkyl groups (or a phenyl group) as provided above.

Some specific examples of carboxylic acids or esters thereof include:

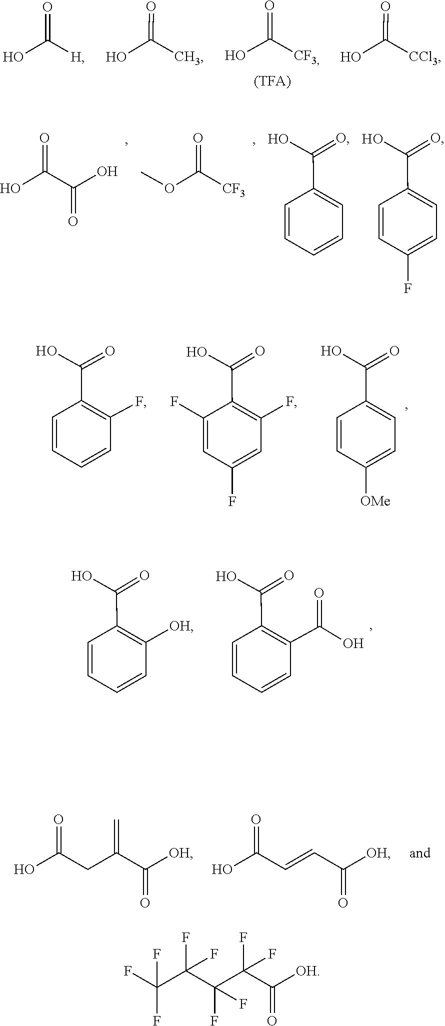

The base may alternatively complex with a sulfonic acid having the general formula R—SO₃R^{e} (where R and R^{e} are as defined above and any specific embodiments thereof), such as:

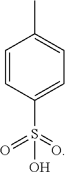

Esters of any of the foregoing acids (i.e., where one or more acidic hydrogen atoms are substituted with one or more R^{d} alkyl groups) are considered herein. Moreover, any base described above may be combined with any acid described above to provide a catalyst. In some embodiments, any base described above, such as TBD or mTBD, may be combined with any of the exemplary acids provided above (e.g., TFA) to result in a catalyst.

The catalyst described above can be produced by combining the base and acid components (i.e., according to Formulas (1) and (2), respectively) under conditions where the two components form a complex with each other. Typically, the two components are combined, in the absence or presence of a solvent, and heated to a temperature of 40-80° C. or more particularly 50-70° C., or about 60° C., for a period of time of 15-45 minutes, more typically about 30 minutes.

The catalyst is typically included in the reaction system in a lower molar amount compared to the molar amount of polymer waste, e.g., no more than 0.25X, 0.2X, 0.1X, or 0.05X, wherein X represents the molar amount of the polymer waste. In some embodiments, after the reaction is complete or near complete, the crude product is mixed with an excess of water to remove excess protic molecule and catalyst from breakdown products.

In particular embodiments, the functionalized alcohol molecule (2) is a hydroxy alkene molecule of the formula:

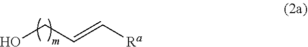

and hence, the terephthalate difunctionalized monomer (3) is a dialkene monomer having the following formula:

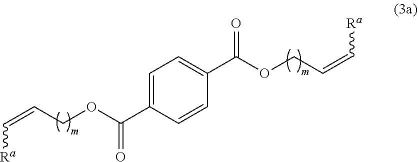

wherein: R^{a} is selected from H and linear, branched, or cyclic alkyl groups containing 1-6 carbon atoms (or more particularly, methyl or ethyl); m is an integer from 1-12 (or more particularly, 1-6, or 1-4) as defined earlier above; and the wavy line connecting to R^{a} indicates that R^{a} may be in a cis or trans position.

Some particular examples of compounds within the scope of (3a) include:

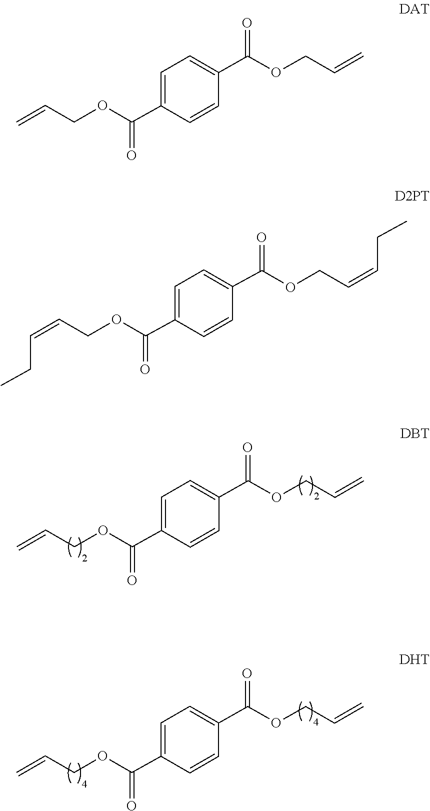

In some embodiments, the monomer (3), as shown in Scheme 1, is polymerized by subjecting the monomer (3) to a polymerization reaction. The polymerization may be achieved by, for example, reaction of the monomer (3) with a reactive crosslinking molecule, by copolymerization with another bifunctional monomer, or by self-polymerization. For example, when R in monomer (3) is or includes an alkenyl group, the alkenyl groups may undergo a thiolene polymerization reaction by crosslinking the alkenyl groups in monomer (3) with a dithiol, trithiol, or tetrathiol molecule; or the alkenyl groups in the monomer (3) may undergo self-polymerization by, e.g., vinyl addition or acyclic diene metathesis (ADMET); or the alkenyl groups in the monomer (3) may undergo vinyl addition copolymerization or ADMET copolymerization when the monomer (3) is polymerized in the presence of another alkene-containing monomer, such as styrene, divinylbenzene, or methacrylate. As another example, when R in monomer (3) is or includes an alkynyl group, the alkynyl groups may undergo a thiol-yne “click” polymerization reaction with a dithiol, trithiol, or tetrathiol molecule, or the alkynyl groups may crosslink with a diazide molecule via azide-alkyne click chemistry to form triazole linkages, as well known in the art. As another example, when R in monomer (3) is or includes an azide group, the azide groups may crosslink with a di- or tri-alkyne molecule via azide-alkyne click chemistry to form triazole linkages, as well known in the art. As another example, when R in monomer (3) is or includes nitrile (CN), the nitrile groups may dimerize or trimerize under suitable conditions, as well known in the art, to form a polymer containing diazine or triazine linkages. As another example, when R in monomer (3) is or includes aldehyde (CHO), the aldehyde groups may crosslink with a diamine molecule to form a polymer containing imine linkages, as well known in the art. As another example, when R in monomer (3) is or includes a terminal amino group (e.g., C(O)NHR^{b}, NHC(O)NHR^{b}, or OCONHR^{b}), the terminal amino groups may crosslink with a dicarboxylic acid, diester, or diisocyanate molecule to form a polymer containing amide, urea, or carbamate linkages. As another example, when R in monomer (3) is or includes a SR^{b} group (where R^{b} is an alkyl group containing 1-3 carbon atoms or a protecting group, such as benzyl, t-butyl, or nitrobenzyl), the R^{b} groups may be removed after deconstruction to permit polymerization by disulfide bond formation, copolymerization with di-alkenes, or crosslinking with tri-alkenes. As another example, when R in monomer (3) is or includes a SSR^{b} group, the SSR^{b} groups may polymerize via disulfide bond exchange. As another example, when R in monomer (3) is or includes a B(OH)₂ group, the B(OH)₂ groups may crosslink with a dihalo or ditriflate hydrocarbon molecule by Suzuki-Miyaura cross-coupling, as well known in the art, particularly when the B(OH)₂ groups are bound to an aryl, alkenyl, or alkynyl group also present in R.

In particular embodiments, the present disclosure is directed to a process of polymerizing a dialkene monomer according to Formula (3a) by an acyclic diene metathesis (ADMET) polymerization process, wherein the ADMET polymerization process is well known in the art.

The ADMET polymerization process is described in, for example, J. T. Patton et al., Macromolecules 25, 3862-3867, 1992, the contents of which are herein incorporated by reference.

The ADMET polymerization process of a dialkene monomer according to Formula (3a) is depicted in the following reaction scheme:

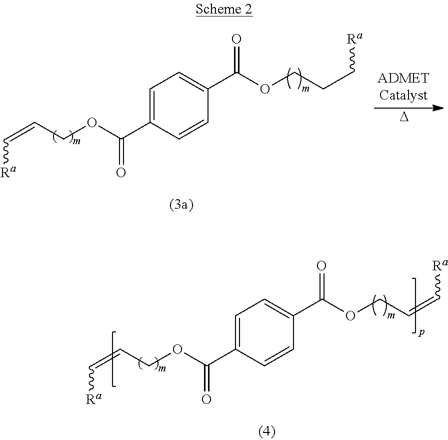

As well known, the ADMET polymerization process employs a Mo-based or Ru-based catalyst under mild elevated temperature conditions (e.g., 40-80° C.) to convert the dialkene monomer to the metathesized polymer. For example, in some embodiments, ADMET polymerization of a dialkene monomer (3a) is achieved by using 2:1 v/v % monomer/solvent in a suitable organic solvent (e.g., 1,2,4-trichlorobenzene) at about 60° C. under dynamic vacuum using G1 catalyst (RuCl₂(═CHPh)(PCy₃)₂) or G2 catalyst (RuCl₂(═CHPh)(H₂IMes)(PCy₃)) with catalyst loadings ranging from 0.2-1 mol %.

In the above Scheme 2, R^{a} is selected from H and alkyl groups containing 1-6 carbon atoms (or more particularly, methyl or ethyl), as described above; m is an integer from 1-6 (or more particularly, 1-4), as described above; and p is at least 10, 20, 30, 40, or 50. The wavy line connecting to R^{a} indicates that R^{a} may be in a cis or trans position. Notably, the ADMET reaction may or may not be conducted in the presence of one or more additional alkene molecules (e.g., styrene, divinylbenzene, or methacrylate) to produce a mixed metathesis product.

In some embodiments, the method further includes subjecting the polymer (4) to a retro-ADMET process in the presence of the hydroxy alkene molecule (2a), ethylene gas, or a mono-functional alkene (e.g., 1-hexene) to afford the dialkene monomer (3a). The retro-ADMET depolymerization process uses the same catalysts and conditions as used for ADMET and is well known in the art. The retro-ADMET process can be used to provide a closed-loop cycle in which ADMET is used to produce the polymer (4) which may be subsequently deconstructed with retro-ADMET to produce a monomer (3a), which can then subsequently be polymerized into the same or other polymer.

A representative retro-ADMET depolymerization process of a polymer (4) is depicted in the following reaction scheme:

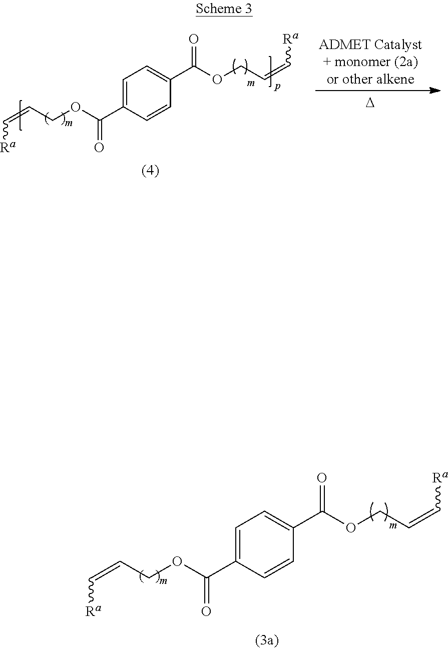

In separate embodiments, the method may also include hydrogenating the polymer (4) to produce a hydrogenated version of polymer (4) in which carbon-carbon double bonds are converted to carbon-carbon single bonds. Hydrogenation processes of unsaturated polymers are well known in the art, and any such process may be used herein. For example, as well known, the unsaturated polymer may be contacted with a source of hydrogen in the presence of a catalyst (which often includes a transition metal, such as Ni, Pd, Pt, Ru, or Rh), optionally under elevated temperature and/or pressure conditions, to effect hydrogenation of the polymer. In other embodiments, the unsaturated polymer may be contacted with a hydrazinyl compound (e.g., p-toluenesulfonyl hydrazide) in a suitable solvent at an elevated temperature (e.g., 120-180° C.) to effect hydrogenation of the polymer.

A representative hydrogenation process of a polymer (4) is depicted in the following reaction scheme:

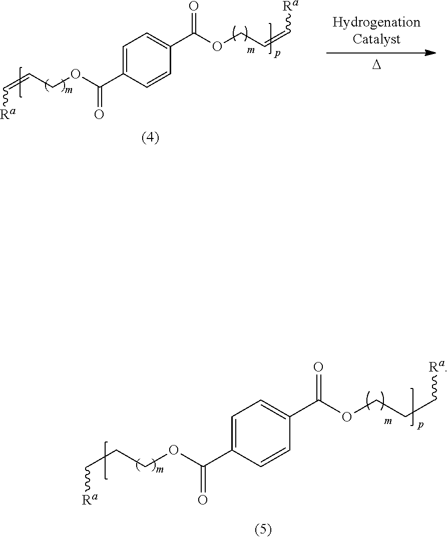

Examples have been set forth below for the purpose of illustration and to describe certain specific embodiments of the invention. However, the scope of this invention is not to be in any way limited by the examples set forth herein.

### Examples

### Synthetic Procedures

Representative synthetic procedures for producing α,ω-dialkenenyl terephthalate monomers starting from terephthaloyl chloride

Synthetic procedure for di(hex-5-en-]-yl) terephthalate (DHT). An oven-dried 2-necked 500 mL round bottom flask containing a magnetic stirring bar was charged with terephthaloyl chloride (14 g, 6920 mmol). The flask was placed under Ar atmosphere via three vacuum-backfill cycles. To the flask was added 200 mL of CH₂Cl₂ and triethylamine (20 mL mL, 140 mmol) under Ar flow. The flask was submerged in an ice bath. 5-hexen-1-ol (16 mL, 130 mmol) was then added dropwise via syringe at 0° C. under Ar. After stirring for 2 h, the ice bath was removed and the reaction mixture allowed to warm to room temperature and stirred overnight under Ar. The reaction mixture was poured into a separatory funnel and washed with 1M HCl soln. (2×50 mL) and saturated NaCl soln. The organic layer was dried over Na₂SO₄ and concentrated in vacuo. The crude residue was purified by flash column chromatography, eluting with 5 v/v % EtOAc in hexanes, to afford DHT (19 g, 86% yield) as a colorless oil. ¹H NMR (400 MHz, CDCl₃): δ 8.10 (s, 4H), 5.82 (m, 2H), 5.01 (dd, 4H), 4.35 (t, 4H), 2.14 (q, 4H), 1.80 (p, 4H), 1.56 (p, 4H). ¹³C NMR (400 MHz, CDCl₃): δ 165.88, 138.27, 134.17, 129.50, 114.98, 65.35, 33.30, 28.11, 25.29.

Synthetic procedure for di(but-3-en-1-yl) terephthalate (DBT). DBT was prepared from terephthaloyl chloride and 3-buten-1-ol using the same procedure as DHT. The crude residue was purified by flash column chromatography, eluting with 10 v/v % EtOAc in hexanes, to afford DBT (9.4 g, 78% yield) as a colorless oil. ¹H NMR (400 MHz, CDCl₃): δ 8.09 (s, 4H), 5.82 (m, 2H), 5.00 (m, 4H), 4.35 (t, 4H), 2.14 (q, 4H), 1.80 (p, 4H), 1.55 (p, 4H). ¹³C NMR (400 MHz, CDCl₃): δ 165.88, 138.27, 134.17, 129.50, 114.98.40, 65.35, 33.30, 28.11, 25.29.

Synthetic procedure for di((Z)-pent-2-en-1-yl) terephthalate (D2PT). D2PT was prepared from terephthaloyl chloride and cis-2-penten-1-ol using the same procedure as DHT. The crude residue was purified by flash column chromatography, eluting with 10 v/v % EtOAc in hexanes, to afford D2PT (5.1 g, 73% yield) as a colorless oil. ¹H NMR (400 MHz, CDCl₃): δ 8.10 (s, 4H), 5.68 (m, 4H), 4.90 (d, 4H), 2.20 (p, 4H), 1.03 (t, 6H). ¹³C NMR (400 MHz, CDCl₃): δ 165.77, 137.47, 134.10, 129.56, 122.40, 61.19, 21.01, 14.13.

Representative synthesis of α,ω-dialkenenyl terephthalate monomers via TBD/TFA-catalyzed deconstruction of pristine PET

Synthetic procedure for di(hex-5-en-]-yl) terephthalate (DHT). A thick-walled glass tube containing a magnetic stirring bar was charged with pristine PET pellets (0.50 g, 2.6 mmol), 5-hexen-1-ol (6.0 mL, 50 mmol), and TBD/TFA catalyst (33 mg, 0.13 mmol). The vessel was sealed with a Teflon screw cap and the reaction mixture was heated to 180° C. in an oil bath for 16 h. After cooling, the reaction mixture was diluted with CH₂Cl₂, filtered to remove the precipitated solids, and concentrated in vacuo. The crude residue was purified by flash column chromatography, eluting with 5 v/v % EtOAc in hexanes, to afford DHT (0.60 g, 92% yield) as a colorless oil. ¹H NMR (400 MHz, CDCl₃): δ 8.10 (s, 4H), 5.82 (m, 2H), 5.01 (dd, 4H), 4.35 (t, 4H), 2.14 (q, 4H), 1.80 (p, 4H), 1.56 (p, 4H). ¹³C NMR (400 MHz, CDCl₃): δ 165.88, 138.27, 134.17, 129.50, 114.98, 65.35, 33.30, 28.11, 25.29.

Synthetic procedure for diallyl terephthalate (DAT). DAT was prepared from pristine PET pellets and allyl alcohol using the same procedure as DHT. The crude residue was purified by flash column chromatography, eluting with 10 v/v % EtOAc in hexanes, to afford DAT (0.30 g, 61% yield) as a colorless oil. ¹H NMR (400 MHz, CDCl₃): δ 8.13 (s, 4H), 6.05 (m, 2H), 5.35 (dd, 4H), 4.85 (d, 4H). ¹³C NMR (400 MHz, CDCl₃): δ 165.46, 134.01, 131.90, 129.63, 118.70, 66.0.

Synthetic procedure for di((Z)-pent-2-en-1-yl) terephthalate (D2PT). D2PT was prepared from pristine PET pellets and cis-2-penten-1-ol using the same procedure as DHT. The crude residue was purified by flash column chromatography, eluting with 5 v/v % EtOAc in hexanes, to afford D2PT (0.52 g, 87% yield) as a colorless oil. ¹H NMR (400 MHz, CDCl₃): δ 8.10 (s, 4H), 5.67 (m, 4H), 4.89 (d, 4H), 2.19 (p, 4H), 1.03 (t, 6H). ¹³C NMR (400 MHz, CDCl₃): δ 165.78, 137.58, 134.10, 129.56, 122.40, 61.19, 21.01, 14.13.

Synthetic procedure for di(but-3-en-1-yl) terephthalate (DBT). DBT was prepared from pristine PET pellets and 3-buten-1-ol using the same procedure as DHT. The crude residue was purified by flash column chromatography, eluting with 10 v/v % EtOAc in hexanes, to afford DBT (0.41 g, 76% yield) as a colorless oil. ¹H NMR (400 MHz, CDCl₃): δ 8.09 (s, 4H), 5.86 (m, 2H), 5.14 (dd, 4H), 4.40 (t, 4H), 2.54 (q, 4H). ¹³C NMR (400 MHz, CDCl₃): δ 165.77, 134.10, 133.85, 129.53, 117.54, 64.39, 33.11.

Representative synthesis of α,ω-dialkenenyl terephthalate monomers via TBD/TFA-catalyzed deconstruction of post-consumer PET: Synthesis of di(hex-5-en-1-yl) terephthalate (DHT)

PET post-consumer waste was prepared for deconstruction by cutting a used PET water bottle into small clippings. A thick-walled glass tube containing a magnetic stirring bar was charged with these post-consumer PET clippings (0.50 g, 2.6 mmol), allyl alcohol (6.0 mL, 50 mmol), TBD/TFA catalyst (33 mg, 0.13 mmol) and additional waste clippings of PS and PP. The vessel was sealed with a Teflon screw cap and the reaction mixture was heated to 180° C. in an oil bath for 16 h. After cooling, the reaction mixture was diluted with CH₂Cl₂, centrifuged to remove the precipitated solids, and concentrated in vacuo. The crude residue was purified by flash column chromatography, eluting with 5 v/v % EtOAc in hexanes, to afford DHT (0.70 g, 93% yield) as a weakly blue oil. Spectral data were consistent with DHT prepared from pristine PET or from terephthaloyl chloride.

Representative ADMET polymerization procedure

A Schlenk tube containing a magnetic stirring bar was charged with G2 catalyst(0.13 g, 0.15 mmol). The tube was sealed with a rubber septum and placed under Ar atmosphere via three vacuum-backfill cycles. DHT (5.0 g, 15 mmol) was mixed with 2 mL of 1,2,4-trichlorobenzene in a 20 mL vial, and the contents of the vial were transferred to the Schlenk tube using a syringe. The tube was submerged into an oil bath pre-heated to 60° C. Vigorous ethylene gas evolution was observed almost immediately. The reaction mixture was stirred for 10 min under Ar atmosphere, after which the tube was opened to vacuum for ca. 2 s to remove the evolving ethylene gas. Vacuum was applied periodically over the next 20 min until the viscosity of the reaction mixture had increased sufficiently to prevent splashing. The reaction mixture was then stirred at 60° C. under vacuum overnight. After cooling, the Schlenk tube was filled with Ar and a mixture of 2 mL of CH₂Cl₂ and 1 mL of ethyl vinyl ether was added via syringe to quench the polymerization. The reaction mixture was stirred for an additional 30 min under Ar and was then poured into MeOH to precipitate the polymer. The resulting viscous, light-brown residue was dried under vacuum at 100° C. for at least 24 h to afford poly(DHT).

Representative polymer depolymerization via Retro-ADMET

A 50 mL round bottom flask containing a magnetic stirring bar was charged with poly(DHT) (1.5 g, 4.9 mmol), G2 (42 mg, 0.049 mmol), and 12 mL of 1-hexene. The flask was equipped with a Findenser and then submerged into an oil bath pre-heated to 60° C. The reaction mixture was stirred at 60° C. for 2 h. After cooling, the reaction was quenched by addition of 20 drops of ethyl vinyl ether. The crude mixture was concentrated in vacuo and small molecule products isolated via flash column chromatography, eluting with 5 v/v % EtOAc in hexanes, to give the deconstruction mixture as a colorless oil (1.45 g, 70% yield).

Representative polymer hydrogenation using tosyl hydrazine

A 250 mL round bottom flask containing a magnetic stirring bar was charged with poly(DHT) (3.6 g, 12 mmol), tosyl hydrazine (11 g, 59 mmol), N,N-diisopropylethylamine (23 mL, 65 mmol), and 100 mL of xylenes. The flask was equipped with a Findenser. The flask was submerged into an oil bath pre-heated to 150° C. and the reaction mixture was heated at reflux overnight. After cooling, the reaction mixture was poured into MeOH to precipitate the polymer. The precipitated solids were collected via vacuum filtration, washed with a large portion of MeOH, and dried under vacuum at 100° C. for at least 24 h to afford poly(DHT-H).

Representative polymer hydrolysis using NaOH

A 20 mL vial containing a magnetic stirring bar was charged with poly(DHT) (ca. 0.1 g). To the vial was added 10 mL of a 5 w/v % solution of NaOH in 60 v/v % EtOH/H₂O. The suspension was stirred at 80° C. for 16 h. After cooling, the solution was extracted with CH₂Cl₂. The CH₂Cl₂ layer was dried over Na₂SO₄and concentrated in vacuo to afford the diol product.

### DISCUSSION

PET Deconstruction to α,ω-Dialkenenyl Terephthalates

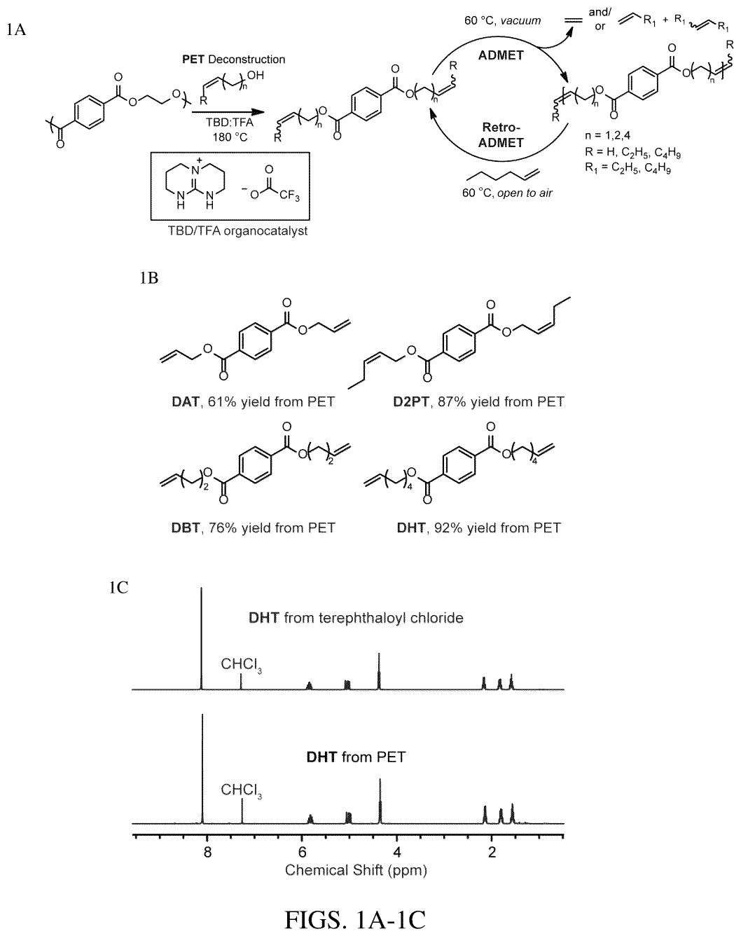

To demonstrate the concept that PET can be transformed into high-value aromatic building blocks beyond its constituent monomers, ω-unsaturated alcohols were selected as transesterification partners for organocatalyzed PET deconstruction. FIG. 1A is schematic showing the PET deconstruction process. PET pellets (M_{w}=40 kg mol-) were suspended in excess alcohol (20 equiv.) in the presence of 5 mol % 1:1 TBD/TFA catalyst and heated at 180° C. in sealed pressure tubes for 16 h. After cooling, the α,ω-dialkene terephthalate products were isolated from the crude reaction mixtures via silica gel filtration and vacuum drying, affording highly pure compounds with yields that ranged from 61-92%. The compounds and their yields are shown in FIG. 1B. The obtained yields were proportional to the boiling point of the alcohol used for deconstruction, and thus, the apparent equilibria of the transesterification reactions. Spectral data for the deconstruction products, as shown in FIG. 1C, were consistent with those measured for reference compounds synthesized from terephthaloyl chloride. Deconstruction using hexen-1-ol was also successful on post-consumer PET waste that contained dyes and other additives, providing DHT with a similar yield (89%) compared with pristine PET, and on a waste plastic mixture containing PET, PS, and PP (93% yield. Diallyl terephthalate (DAT), an important commercial plasticizer and resin curative, was prepared directly from PET using this method. The other dialkenes (D2PT, DBT, and DHT) were shown to be amenable to polymerization via ADMET and may also have interesting applications beyond olefin metathesis such as in cationic, radical, or thiol-ene formulations or as building blocks in organic synthesis.

ADMET Polymerization of α,ω-Dialkenenyl Terephthalate Monomers

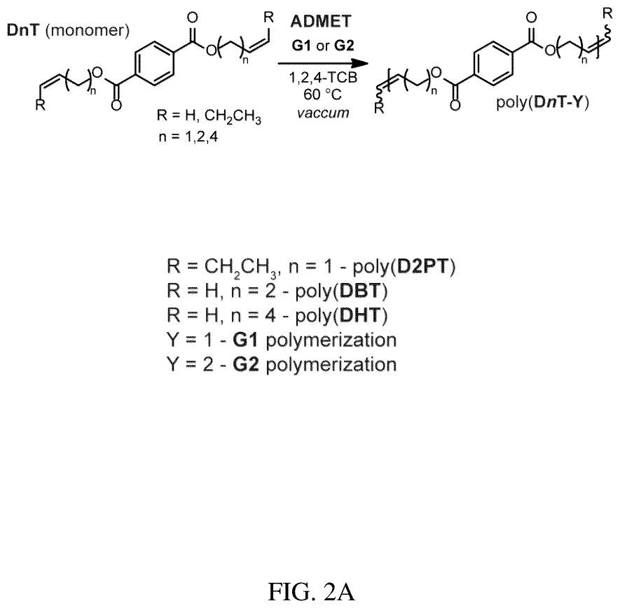

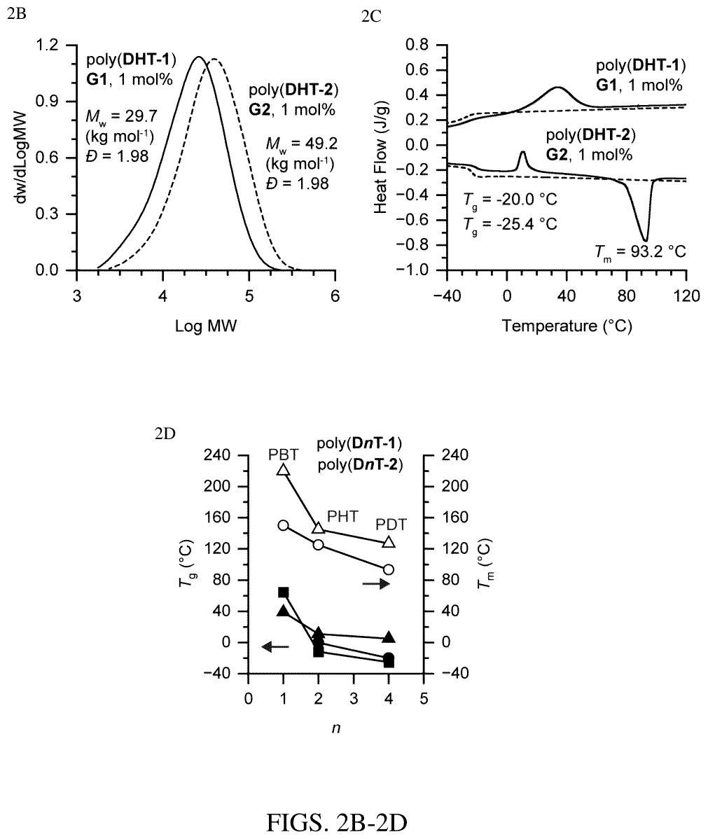

Initial experiments attempted ADMET polymerization of DHT, which has been successfully polymerized using both Mo- and Ru-based metathesis catalysts. FIG. 2A is a schematic of ADMET polymerization of DnT monomers. ADMET polymerization of DHT at 2:1 v/v % monomer/solvent in 1,2,4-trichlorobenzene at 60° C. under dynamic vacuum was successfully achieved using G1 (RuCl₂(═CHPh)(PCy₃)₂) or G2 (RuCl₂(═CHPh)(H₂IMes)(PCy₃)) catalyst, with catalyst loadings ranging from 0.2-1 mol %. DHT polymerization by ADMET reproducibly provided polymers with moderate molecular weights (MWs) (M_{w} ranging from ca. 30 kg mol⁻¹ for G1 polymerizations to ca. 45 kg mol⁻¹ for G2 polymerizations). As shown by the overlaid MW distributions in FIG. 2B, dispersities, D, were near 2 in all cases, consistent with a step-growth mechanism of polymerization and high conversion. Significantly, polymerizations at low catalyst loadings (0.2 mol %) produced similar polymers to those with more catalyst, highlighting the potential for larger scale up.

It should be noted that ADMET polymerization produces ethylene or other small molecule alkene condensates that must be actively removed to drive the reaction equilibrium towards high molecular weight polymers, and this fact must be accounted for in reactor design. As shown by the stacked DSC traces in FIG. 2C, poly(DHT) synthesized using G1, poly(DHT-1), was semi-crystalline with T_{g}=−20.0° C. and Tₘ=93.2° C., while poly(DHT) obtained from G2 polymerization, poly(DHT-2), was amorphous with T_{g}=−25.4° C. In addition to T_{g} and Tₘ, cold crystallization was observed as an exothermic transition at ca. 10° C. in the DSC trace of poly(DHT-1) on the second heating cycle (FIG. 2C). Hsu et al. showed that olefin stereochemistry strongly modulates the thermal properties of ADMET polymers (T.-W. Hsu et al., Nat. Chem. 15, 14-20, 2023). Here, the observed differences in thermomechanical properties could not be attributed to stereochemical effects particularly since poly(DHT-1) and poly(DHT-2) possessed similar olefin (cis/trans) compositions. Instead, the differences in poly(DHT) thermomechanical behavior may be attributed to olefin isomerization by G2; i.e., the movement of C═C bonds along the polymer backbone, as has been observed in related systems (e.g., B. Schmidt, EurJOC, 1865-1880. https://doi.org/10.1002/ejoc.200300714). Olefin isomerization during ADMET produces a broad distribution of repeat unit structures possessing different numbers of methylene units between each terephthalate group. Indeed, additional downfield olefinic signals were present in the ¹H NMR spectra of poly(DHT-2) that were assigned to products of olefin migration towards the ester groups in the polymer backbone. Defects along the polymer backbone produced by olefin isomerization likely influenced the bulk polymer microstructures and morphologies of poly(DHT-1) and poly(DHT-2) similar to previous reports (e.g., K. J. Arrington et al., Macromolecules 49, 3655-3662, thereby affecting their ultimate thermal and mechanical properties. Attempts to suppress olefin metathesis using 1,4-benzoquinone or acetic acid dramatically reduced the MW of the resulting polymers and were not further pursued.

Next experiments investigated the polymerization of α,ω-diene monomers possessing different numbers of methylene units between the ester and alkene functionalities. It was herein found that DAT was inert to ADMET polymerization regardless of the catalyst used. Surprisingly, D2PT, which also possesses a single methylene unit in its spacer, was oligomerized during both G1—and G2-initiated ADMET reactions, providing indirect access to poly(DAT). The enhanced activity in D2PT ADMET polymerization likely resulted from the relatively increased stability of the propylidene intermediate generated by its metathesis compared with Ru methylidene as has been shown by Hoveyda and coworkers (C. Xu et al., J. Am. Chem. Soc. 139, 10919-10928. 10.1021/jacs.7b06552). This hypothesis is supported by the fact that DAT underwent nearly quantitative conversion to terephthalic acid in the presence of G2 catalyst over 24 h at 60° C., which presumably occurred through a de-allylation pathway mediated by decomposed Ru species (M. Kitamura et al., J. Org. Chem., 67, 4975-4977. 10.1021/jo0202810). The foregoing result demonstrates the synthesis of poly(DAT) via alkene polymerization, thus providing a metathesis route to obtain polybutylene terephthalate (PBT) and derivatives thereof via hydrogenation. PBT is a high-value engineering thermoplastic and potentially represents an ideal platform for incorporation of upcycled material.

DBT was also amenable to ADMET polymerization; however, ADMETs of D2PT or DBT were limited by the solubility of their growing oligomers, as their reaction mixtures quickly solidified at 60° C. In fact, number-average molecular weights, Mₙ, and degrees of polymerization, Xₙ, of the polymers produced using these monomers with either G1 or G2 catalyst scaled with increasing methylene spacer length, n, corresponding with the relative solubility of their oligomers in the reaction media. The SEC traces for poly(DBT-2) and poly(D2PT-1) were bimodal, further indicating that these samples contained oligomers of discreet lengths. Polymerization at higher temperatures or in different solvents may increase the achievable MW but was not attempted due to concerns regarding olefin isomerization, catalyst decomposition, and solvent evaporation. As shown by the data in FIG. 2D, in contrast to the MW trends, both glass transition temperatures T_{g}, and melting temperatures Tₘ, decreased with increasing n. This decrease is consistent with the thermal behavior of their fully saturated analogs poly(butylene terephthalate) (PBT), poly(hexamethylene terephthalate) (PHT), and poly(decamethylene terephthalate) (PDT) (FIG. 2D).

Mechanical Characterization of poly(DHT)

Solvent casting or melt pressing of poly(DHT-1) afforded films that were too brittle for mechanical testing. Likewise, films prepared from poly(DHT-2) were very sticky and could not be delaminated from the various sample preparation media. Films were obtained from solvent casting of poly(DHT) with different molecular weights prepared using G1 or G2 catalyst. Some reports on step-growth materials have demonstrated a strong dependence of mechanical properties on MW, with an apparent cutoff existing at M_{w}˜70 kg mol⁻¹ in some systems only above which “useful” mechanical properties are obtained (e.g., M. Piccini et al., Polym. Chem. 11, 2681-2691. 10.1039/C9PY01809C). To investigate MW effects in the present system, DHT polymerization conditions were modified to target high MW, including higher dilution (1:1 v/v % monomer/solvent instead of 2:1 v/v %), higher temperature (75° C.), a longer reaction time, and multiple catalyst additions.

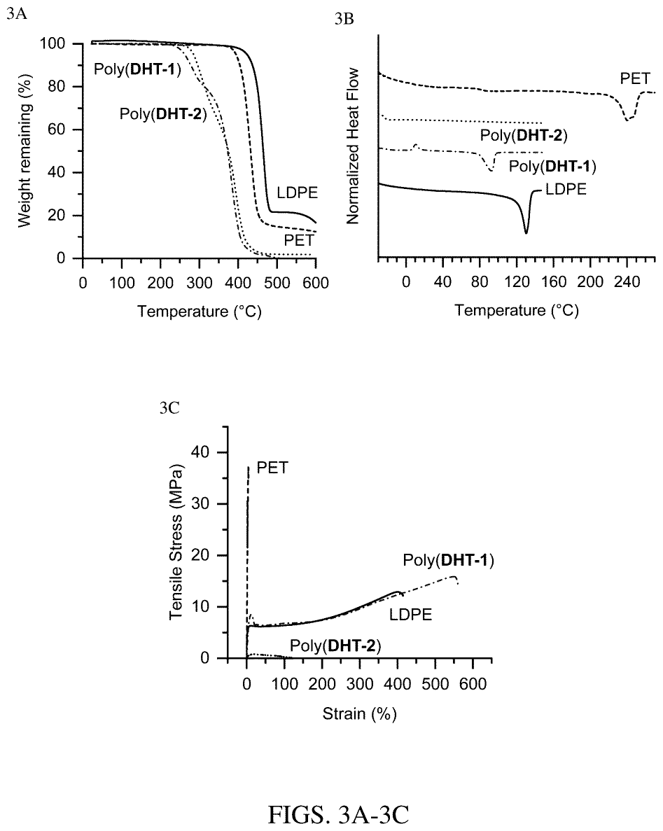

Consistent with previous reports on ADMET polymerization, it was herein found that reaction times of at least 6 d were required to achieve M_{w}>70 kg mol⁻¹ (H. Li et al., Polym. Int. 66, 7-12. doi.org/10.1002/pi.5188). High MW poly(DHT), prepared using G1 or G2 catalyst, formed free-standing films, with poly(DHT-1) amenable to casting films as thin as 20 μm. As shown in FIG. 3C, both samples exhibited high ductility reflected in their large elongation at break, ε_{b}, values. While high MW poly(DHT-2) was rubbery, its G1-derived analog, poly(DHT-1), was tough with an ultimate strength of ca. 16 MPa and ε_{b}˜550% (FIG. 3C). These data demonstrate a significant transformation of mechanical properties from the starting PET, wherein a strong but insoluble thermoplastic with limited recyclability was converted into a tough, highly soluble, and processable polymer with intrinsic recyclability possessing mechanical properties that were comparable to commodity materials such as LDPE. As indicated by the data in FIGS. 3A and 3B, while the thermal stability and melting temperature of poly(DHT) were reduced compared with PET or LDPE, likely due to the presence of backbone unsaturation, these properties can be improved by hydrogenation (vide infra). The thermomechanical performance may be optimized by further increasing MW, tuning the length of the methylene spacer between terephthalate units, or by better controlling backbone regio- and stereochemistry to obtain materials comparable to recent prominent PE alternatives.

Deconstruction of poly(DHT) Using Retro-ADMET

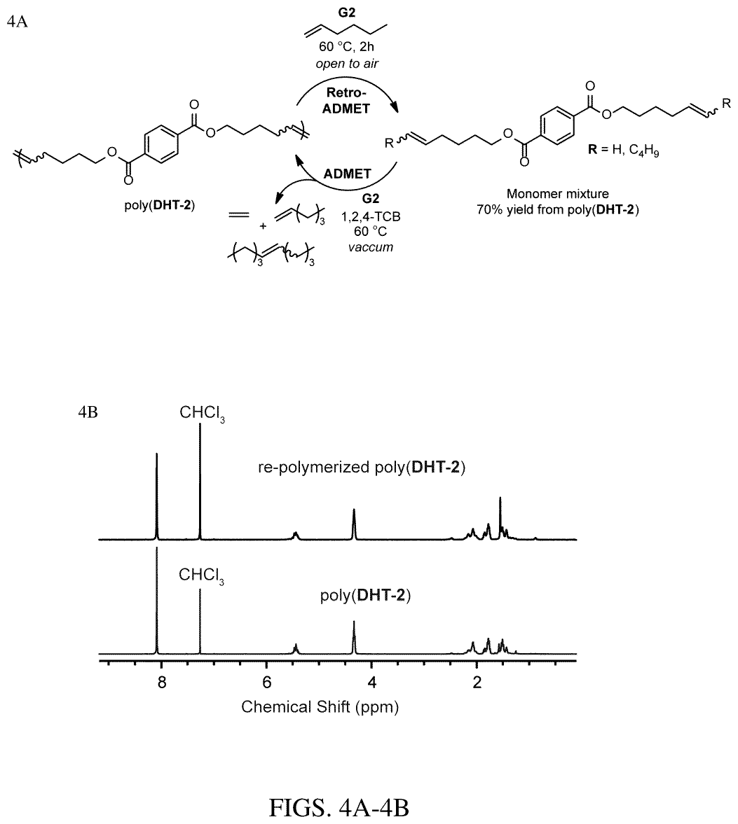

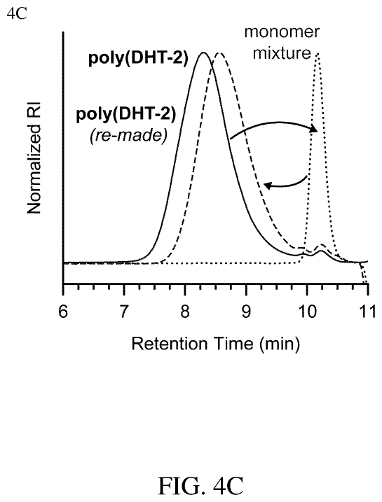

Polymerization of α,ω-dialkenenyl terephthalate monomers by ADMET afforded semi-aromatic polyesters with backbone unsaturations between each repeat unit. These backbone olefins offered the possibility of further modification through hydrogenation or thiol-ene modification, for example. They can also be targeted for polymer deconstruction using Retro-ADMET (cross-metathesis with lower olefins). Towards this end, poly(DHT-2) was treated with excess 1-hexene (20 equiv relative to backbone olefins) and 2 mol % G2 catalyst to regenerate the DHT monomer. A schematic of the retro-ADMET process is provided in FIG. 4A. The deconstruction reaction was conducted at equilibrium in only 2 hours at 60° C. open to air, cleanly generating predominately monomeric products possessing a mixture of terminal and internal alkenes (derived from cross-metathesis with 1-hexene). The monomer mixture was isolated from Ru, 5-decene (cross-metathesis byproduct), oligomers, and residual 1-hexene via silica gel filtration and vacuum drying to afford a colorless, viscous oil amenable to direct re-polymerization.

Poly(DHT-2) polymerized from the deconstruction mixture exhibited a similar ¹H NMR spectrum (FIG. 4B), a MW distribution shifted to a slightly longer retention time (FIG. 4C), M_{w} of ca. 17 kg mol⁻¹ for the re-polymerized sample), and a nearly identical T_{g} value compared with the native material. The decrease in MW following re-polymerization was attributed to: (1) the reduced reactivity of internal alkenes in olefin metathesis reactions compared with the terminal alkenes of DHT; (2) the low volatility of 1-hexene and 5-decene (relative to ethylene)—the major condensation byproducts of ADMET for this monomer mixture; and (3) olefin migration resulting in the formation of allylic alkenes with reduced reactivity. The latter issue is particularly problematic when considering multiple depolymerization/repolymerization cycles since each cycle would presumably produce additional allylic alkenes that may not participate in ADMET polymerization. These issues could potentially be circumvented by using ethylene instead of 1-hexene during the deconstruction reaction and by employing cyclic aminoalkane carbene (CAAC) catalysts in both steps instead of G1 or G2, which dare known to not promote olefin migration reactions. These data demonstrate the potential for closed-loop circularity using metathesis chemistry for both polymerization and deconstruction.

### Hydrogenation of Poly(DHT)

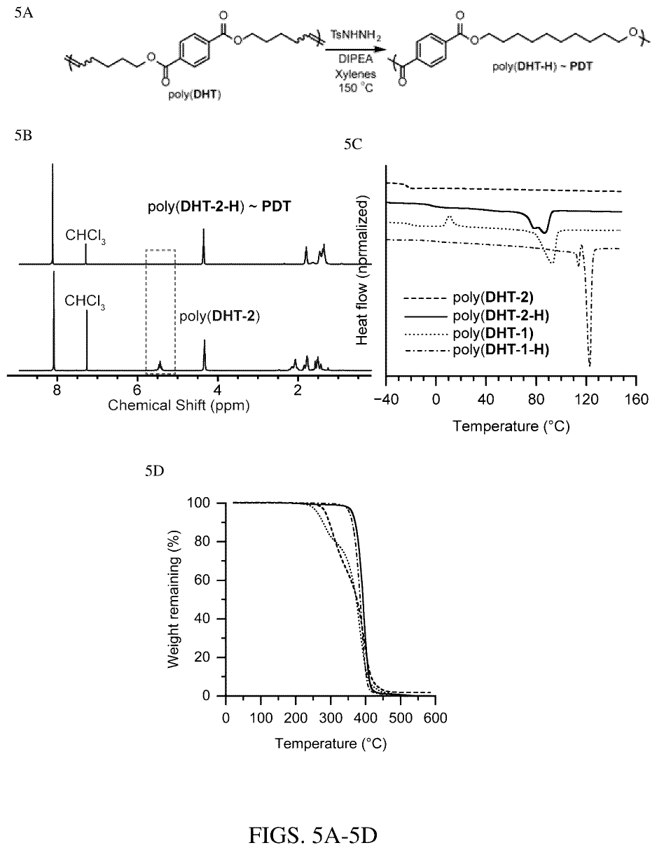

Samples of poly(DHT) were shown to possess significantly different thermomechanical behavior dependent on the catalyst used for their ADMET polymerization. Further experiments aimed to study their saturated analogues, which have a structure similar to poly(decamethylene terephthalate) (PDT). It should be noted that PDT and other long-chain polyalkyl terephthalates have garnered interest for their high biocompatibility and in particular their capability to adhere to endothelial cells (e.g., B. Van de Voorde et al., Eur. Polym. J., 165, 111003. doi.org/10.1016/j.eurpolymj.2022.111003). As shown in the scheme provided in FIG. 5A, saturated polymers were prepared by hydrogenation using tosyl hydrazide. As shown by the data in FIG. 5B, complete conversion of backbone olefins was verified by the disappearance of protons located at ca. 5.5 ppm in the ¹H NMR spectra of the hydrogenated polymers. As shown by the DSC data in FIG. 5C, compared with amorphous poly(DHT-2), its saturated equivalent, poly(DHT-2-H), exhibited semi-crystalline behavior with a broad Tₘ centered on ˜80° C. The thermal behavior of poly(DHT-1) was also altered by hydrogenation, with its original broad Tₘ˜85° C. becoming more narrow and shifting to a higher temperature of −120° C. (FIG. 5C). In both cases, the evolution of thermal behaviors observed following hydrogenation may be attributed to increased structural regularity, since hydrogenation removes geometrically constraining olefins from the polymer backbones, thereby facilitating packing during crystallization. As shown by the TGA data in FIG. 5D, hydrogenation also increased the thermal stability of poly(DHT) regardless of the method of preparation.

To gain further insight into the differences in thermal behavior between polymers prepared using either G1 or G2 catalyst, the hydrogenated polymers were hydrolyzed using 5 wt/v % NaOH in 1:1 v/v % EtOH/H₂O at 80° C. After hydrolysis, the resulting long-chain diols were isolated from terephthalic acid via extraction with CH₂Cl₂ and characterized using ¹H NMR spectroscopy. From the hydrolysis experiment, it was found that the mixture of products obtained from poly(DHT-2-H) contained a significant fraction of 8 and 9 carbon diols, while the hydrolysate from poly(DHT-1-H) was dominated by the expected 1,10-dodecanediol. The data is consistent with the characterization of the polymers prior to hydrogenation, where olefin migration was apparent in their ¹H NMR spectra. As such, poly(DHT-1) can be considered to possess higher regioregularity as compared with poly(DHT-2). Its high regioregularity provides it with good thermomechanical performance as reported above.

In summary, a closed-loop production and recycling approach has herein been demonstrated for unsaturated semi-aromatic polyesters using olefin metathesis chemistry. Significantly, these polymers were prepared from dialkenenyl terephthalate monomers derived from organocatalytic PET deconstruction. The resulting materials possess thermomechanical properties that may be tuned across a broad range (i.e., from amorphous to semi-crystalline) based on monomer and catalyst selection. By increasing molecular weight, free-standing films can be obtained that exhibit good mechanical performance similar to widely manufactured commodity polymers. Of further significance, the polymers can be deconstructed back to monomers under mild conditions using a fast Retro-ADMET approach. This work demonstrates that PET can serve as a feedstock resource to produce value-added small molecules and materials beyond its constituent monomers. While not currently cost competitive with commodity materials, the present results demonstrate the substantial potential of the presently described alternative approach towards circular polymer production, resource conservation, and reduction of CO₂ emissions associated with plastics production.

While there have been shown and described what are at present considered the preferred embodiments of the invention, those skilled in the art may make various changes and modifications which remain within the scope of the invention defined by the appended claims.

What is claimed is:

1. A method of deconstructing and upcycling polyethylene terephthalate (PET) waste into at least one useful breakdown product, the method comprising contacting the PET waste (1) with a catalyst comprising an organic nitrogen-containing base and a carboxylic acid or ester thereof, in the presence of an R-functionalized alcohol molecule (2) under transesterification conditions to produce a terephthalate difunctionalized monomer (3), according to the following reaction scheme: 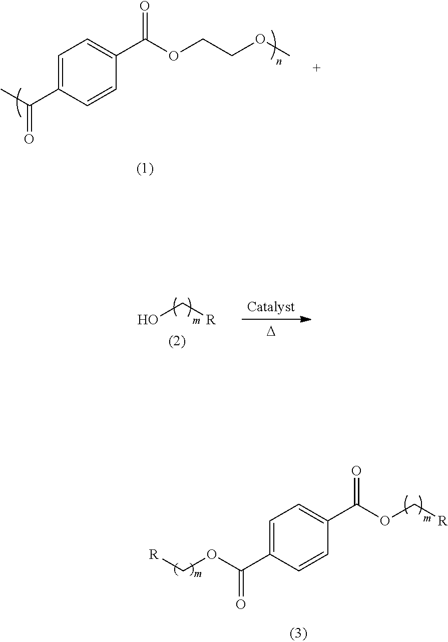
wherein:
R is a functional group selected from the group consisting of alkenyl, alkynyl, N₃, CN, OR^{b}, COOH, CHO, C(O)R^{b}, C(O)NHR^{b}, NHC(O)NHR^{b}, OCONHR^{b}, SR^{b}, SSR^{b}, and B(OH)₂ groups, wherein R^{b} is an alkyl group containing 1-3 carbon atoms;
n is at least 10;
m is an integer from 1-12; and
the symbol Δ denotes an elevated temperature sufficient to promote a transesterification reaction between (1) and (2) in the presence of the catalyst.

2. The method of claim 1, wherein the terephthalate difunctionalized monomer (3) is polymerized by reaction with a reactive crosslinking molecule, by self-polymerization, or by copolymerization with another bifunctional monomer.

3. The method of claim 1, wherein m is an integer from 1-6 or from 1-4.

4. The method of claim 1, wherein the organic nitrogen-containing base is a bicyclic guanidine having the following structure: 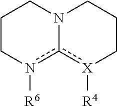
wherein R⁴ and R⁶ are selected from H and CH₃ groups, and the dotted lines represent optional double bonds.

5. The method of claim 4, wherein the bicyclic guanidine is triazabicyclodecene (TBD).

6. The method of claim 1, wherein the catalyst comprises triazabicyclodecene and a carboxylic acid.

7. The method of claim 6, wherein the carboxylic acid is trifluoroacetic acid (TFA).

8. The method of claim 1, wherein the catalyst comprises 1:1 TBD:TFA.

9. The method of claim 1, wherein the elevated temperature is within a range of 150-200° C.

10. The method of claim 1, wherein the elevated temperature is within a range of 170-190° C.

11. The method of claim 1, wherein the functionalized alcohol molecule (2) is a hydroxy alkene molecule of the formula: 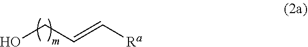
and the terephthalate difunctionalized monomer (3) is a dialkene monomer having the following formula: 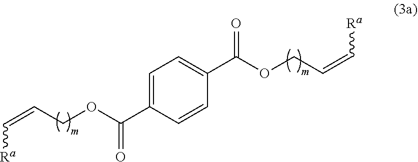
wherein:
R^{a} is selected from H and alkyl groups containing 1-6 carbon atoms;
m is an integer from 1-12; and
the wavy line connecting to R^{a} indicates that R^{a} may be in a cis or trans position.

12. The method of claim 11, wherein m is an integer from 1-6 (or 1-4).

13. The method of claim 11, wherein R^{a} is H.

14. The method of claim 11, wherein R^{a} is an alkyl group containing 1-6 carbon atoms.

15. The method of claim 14, wherein R^{a} is methyl or ethyl.

16. The method of claim 11, further comprising polymerizing the dialkene monomer (3a) by an acyclic diene metathesis (ADMET) polymerization process to produce the following polymer: 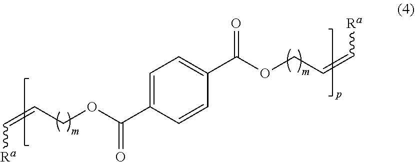
wherein:
R^{a} is selected from H and alkyl groups containing 1-6 carbon atoms;
m is an integer from 1-6;
p is at least 10; and
the wavy line connecting to R^{a} indicates that R^{a} may be in a cis or trans position.

17. The method of claim 16, wherein m is an integer from 1-6 or from 1-4.

18. The method of claim 16, wherein R^{a} is H.

19. The method of claim 16, wherein R^{a} is an alkyl group containing 1-6 carbon atoms.

20. The method of claim 19, wherein R^{a} is methyl or ethyl.

21. The method of claim 16, further comprising subjecting the polymer (4) to a retro-ADMET process in the presence of the hydroxy alkene molecule (2a), ethylene gas, or a mono-functional alkene (e.g., 1-hexene) to afford the dialkene monomer (3a).

22. The method of claim 16, further comprising hydrogenating the polymer (4) to produce a hydrogenated version of polymer (4) in which carbon-carbon double bonds are converted to carbon-carbon single bonds.
